(19) United States
(12) Patent Application Publication
(10) Pub. No.: US 2026/0232234 A1
(43) Pub. Date: Aug. 13, 2026

(54) BIODEGRADABLE HYBRID SILICON POLYMER SENSOR FOR POST OPERATIVE MONITORING

(71) Applicant: Georgia Tech Research Corporation, Atlanta, GA (US)

(72) Inventors: Luke Beardslee, Atlanta, GA (US); Chinaza Ogbonna, Atlanta, GA (US); Nima Ghalichechian, Atlanta, GA (US); Oliver Brand, Atlanta, GA (US); Yuheng He, Atlanta, GA (US)

(21) Appl. No.: 19/533,075

(22) Filed: Feb. 6, 2026

Related U.S. Application Data
(60) Provisional application No. 63/755,457, filed on Feb. 7, 2025.

(51) Int. Cl. A61B 5/1473; A61B 5/145; A61L 31/02; A61L 31/04; A61L 31/06; A61L 31/14

(52) U.S. Cl. CPC A61B 5/14735; A61B 5/14539; A61B 5/14546; A61L 31/022; A61L 31/048; A61L 31/06; A61L 31/148; A61B 2562/125

(57) ABSTRACT

The present disclosure relates generally to biodegradable sensors for biomedical applications, and more particularly to a wirelessly interrogatable, bioresorbable sensor for postoperative monitoring of physiological parameters. A sensor system includes a substrate, an electrode layer disposed on the substrate, the electrode layer including a split-ring resonator coupled to at least one interdigitated capacitor, wherein the at least one interdigitated capacitor is connected in series with the split-ring resonator, a passivation layer disposed on the electrode layer, and a sensing film disposed on the passivation layer, wherein the sensing film is configured to change in thickness in response to an analyte of interest, and wherein a change in thickness of the sensing film causes a shift in a resonance frequency of the sensor system.

### CROSS-REFERENCE TO RELATED APPLICATIONS

This application claims the benefit of U.S. Provisional Application Ser. No. 63/755,457, filed on 7 Feb. 2025, which is incorporated herein by reference in its entirety as if fully set forth below.

### FIELD OF INVENTION

The present disclosure relates to biodegradable sensors for biomedical applications, and more particularly to a wirelessly interrogatable, bioresorbable sensor comprising a split-ring resonator loaded with interdigitated capacitors for postoperative monitoring of physiological parameters such as pH and enzyme activity.

### BACKGROUND

Postoperative complications following abdominal (as well as neck and extremity) surgery represent a significant clinical challenge in modern healthcare. Complications include, but are not limited to a leak at a bowel anastomosis, which occurs when surgical connections between portions of the gastrointestinal tract fail to heal properly, can lead to serious consequences including peritonitis, sepsis, and extended hospital stays. Additional complications include bleeding from a variety of different sources either a raw surface or a vessel, abscess formation, leaking of another anastomosis (i.e., bile duct to small bowel or pancreas to small bowel, and leaking from a ligated structure such as the cystic duct, or a staple line on the stomach, liver or another organ, which may have been removes/resected. Current diagnostic approaches for detecting such complications rely on a combination of clinical assessments, imaging studies such as computed tomography scans, and laboratory tests including white blood cell counts. These diagnostic methods are typically employed after patients begin exhibiting symptoms, which may occur several days following surgery and after a systemic shock response has started.

Wireless sensing technologies have been developed for various biomedical monitoring applications. Radio frequency identification (RFID) systems offer advantages including low power consumption, non-contact data transfer, and cost-effectiveness. Chipless RFID systems encode information using resonance structures and allow remote readout without requiring active electronic components or batteries. Split-ring resonators and interdigitated capacitors have been utilized in sensing applications due to their self-resonance characteristics and sensitivity to changes in surrounding dielectric properties. Biodegradable materials including polycaprolactone substrates and molybdenum electrodes have been explored for implantable medical devices that can be absorbed by the body over time.

However, existing approaches for postoperative monitoring suffer from several limitations. Current diagnostic methods often lack the specificity, sensitivity and speed required for early detection of complications before patients become systemically ill. Inductive coupling techniques used in some wireless sensing systems suffer from limited reading distances and instability against channel changes. Many existing sensors are not fully biodegradable, requiring surgical removal after their monitoring function is complete. Additionally, conventional sensor designs that place interdigitated capacitors within the radiation gap of split-ring resonators cause electric field confinement that limits backscattering signal strength for wireless interrogation at practical distances.

What is needed, therefore, is an improved biodegradable sensor system that enables wireless interrogation at extended distances while maintaining sensitivity to physiological parameters indicative of postoperative complications. Such a system would provide real-time monitoring capability using fully bioresorbable materials that eliminate the need for secondary removal procedures.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

A sensor system can include a substrate. The sensor system can include an electrode layer disposed on the substrate. The electrode layer can include a split-ring resonator coupled to at least one interdigitated capacitor. The at least one interdigitated capacitor can be connected in series with the split-ring resonator. The sensor system can include a passivation layer deposited onto the electrode layer. The sensor system can include a sensing film deposited on the passivation layer. The sensing film can be configured to change in thickness in response to an analyte of interest. A change in thickness of the sensing film can cause a shift in a resonance frequency of the sensor system.

A sensor system for postoperative monitoring can include a biodegradable substrate. The sensor system can include an electrode structure disposed on the biodegradable substrate. The electrode structure can form a resonant circuit including an inductive element and a capacitive element. The capacitive element can include a first interdigitated capacitor and a second interdigitated capacitor positioned on opposite sides of a radiation gap of the inductive element. The sensor system can include a passivation layer disposed over the electrode structure. The sensor system can include a sensing film disposed over the passivation layer. The sensing film can be configured to either degrade or increase in thickness in response to a physiological parameter. Degradation of the sensing film can alter a capacitance of the capacitive element to produce a detectable shift in a resonance frequency of the resonant circuit.

A method of fabricating a biodegradable sensor can include providing a substrate. The method can include depositing an electrode layer on the substrate. The electrode layer can include a split-ring resonator and at least one interdigitated capacitor connected in series with the split-ring resonator. The method can include depositing a passivation layer over the electrode layer. The method can include depositing a sensing film over the passivation layer. The sensing film can be configured to change in thickness in response to an analyte of interest.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a schematic diagram of a biodegradable chipless RFID sensor for pH monitoring, according to examples of the disclosed technology.

FIG. 2A illustrates an equivalent circuit diagram of a sensor system, according to examples of the disclosed technology.

FIG. 2B illustrates a top view of a sensor design having a split-ring resonator with interdigitated capacitors, according to examples of the disclosed technology.

FIG. 3 illustrates an exploded view of a biocompatible sensor system, according to examples of the disclosed technology.

FIG. 4 illustrates an exploded view of a bioresorbable sensor system, according to examples of the disclosed technology.

FIG. 5 is a flow diagram for a method for fabricating a biocompatible sensor, according to examples of the disclosed technology.

FIG. 6 is a flow diagram for a method for fabricating a bioresorbable sensor, according to examples of the disclosed technology.

FIG. 7 illustrates a graph of simulated radar cross section as a function of frequency for different sensing film thicknesses, according to examples of the disclosed technology.

FIG. 8 illustrates a graph of simulated radar cross section amplitude as a function of frequency for different sensing film thicknesses, according to examples of the disclosed technology.

FIG. 9A illustrates a graph of normalized frequency as a function of time for different pH conditions, according to examples of the disclosed technology.

FIG. 9B illustrates a graph of normalized frequency as a function of time for different pH conditions, according to examples of the disclosed technology.

FIG. 9C illustrates a graph of normalized frequency as a function of time for different pH conditions, according to examples of the disclosed technology.

FIG. 9D illustrates a graph of normalized frequency as a function of time for different pH conditions, according to examples of the disclosed technology.

FIG. 10A illustrates a graph of force as a function of distance for mechanical testing of sensing films, according to examples of the disclosed technology.

FIG. 10B illustrates a graph of stress-strain curves for mechanical testing of sensing films, according to examples of the disclosed technology.

FIG. 11 illustrates a bar chart of cell viability results from cytotoxicity testing of sensor film samples, according to examples of the disclosed technology.

FIG. 12 illustrates a graph of degradation rate as a function of trypsin concentration for various protein blend compositions, according to examples of the disclosed technology.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a biodegradable sensor system configured for postoperative monitoring. The sensor system can be wirelessly interrogatable and bioresorbable, comprising a split-ring resonator loaded with interdigitated capacitors. The sensor system can detect physiological parameters such as pH levels and enzyme activity. In some cases, the sensor system can be configured for detection of an anastomotic leak in gastrointestinal surgery applications. An anastomotic leak represents a complication that can occur following gastrointestinal surgery, and early detection of such leaks can facilitate timely clinical intervention. The sensor system can monitor changes in pH levels or enzymatic activity that can indicate the presence of anastomotic leakage, thereby providing a wireless solution for continuous postoperative monitoring without requiring surgical removal of the sensor following use.

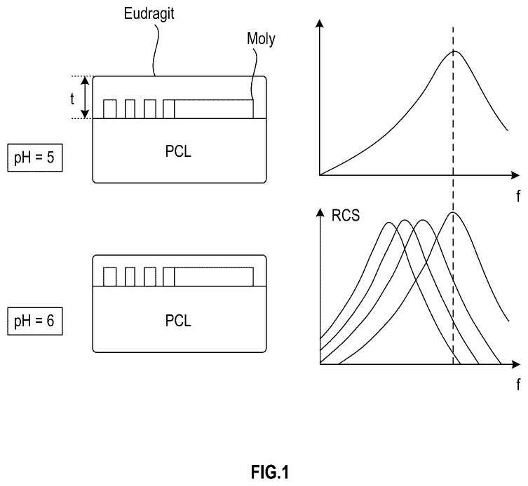

Referring to FIG. 1, a sensor system operates according to a chipless RFID principle where information is encoded in a resonance frequency of the sensor system. The sensor system comprises a sensing film disposed above an electrode structure. The sensing film can comprise Eudragit, which is a pH-sensitive polymer material. The electrode structure can comprise molybdenum (Moly) electrodes arranged in an interdigitated pattern. A substrate can comprise polycaprolactone (PCL) disposed beneath the electrode structure. The sensing film has a thickness dimension denoted as t, and the sensing film is configured to change in thickness in response to an analyte of interest.

With continued reference to FIG. 1, the sensor system operates based on subtractive sensing where the sensing film degrades and is removed in response to the analyte. At a first pH level of pH 5, the sensing film remains intact with a thickness t, and the sensor system exhibits a resonance frequency at a first position as shown in a corresponding radar cross section (RCS) response graph. At a second pH level of pH 6, the sensing film degrades in the solution, resulting in a reduced thickness or complete removal of the sensing film. A change in thickness of the sensing film causes a shift in a resonance frequency of the sensor system, as illustrated by the shifted resonance curves in the RCS response graph corresponding to the pH 6 condition.

The sensor system can operate at around 3.2 GHz in free space. When the sensor system is placed in a liquid solution environment, the sensor system can operate at around 2 GHz. The shift in operating frequency between free space and liquid solution conditions results from changes in the effective permittivity of the surrounding medium. As the sensing film degrades in response to elevated pH levels, the capacitance of the interdigitated electrode structure changes, which produces a detectable shift in the resonance frequency. The RCS response of the sensor system provides a wireless readout mechanism where the resonance frequency shift can be detected remotely without requiring direct electrical connection to the sensor system.

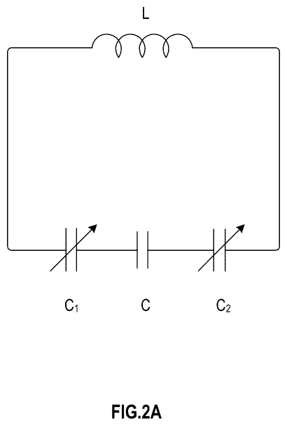

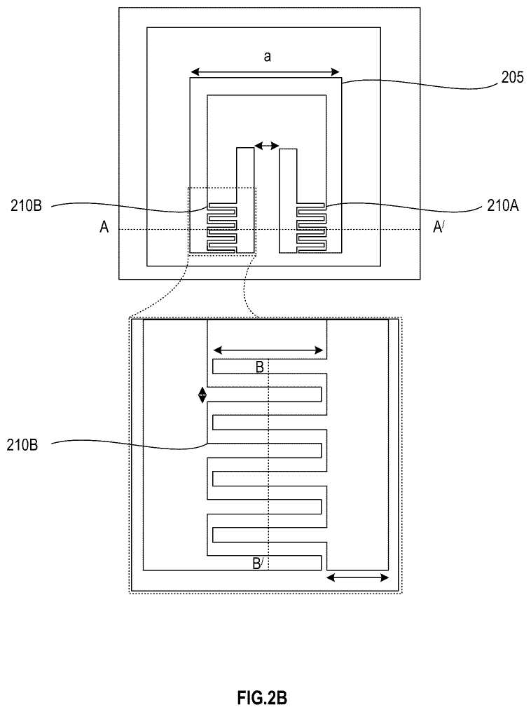

Referring to FIG. 2A, an equivalent circuit diagram represents the sensor system. The circuit comprises an inductor L connected in a loop configuration with three capacitors arranged in parallel at a bottom portion of the circuit. The inductor L represents a split-ring resonator portion of the sensor system, which functions as an inductive element in a resonant circuit. The split-ring resonator can be referred to herein as the inductive element.

With continued reference to FIG. 2A, a first variable capacitor C1 is positioned on a left side of the parallel arrangement, and a second variable capacitor C2 is positioned on a right side of the parallel arrangement. Both the first variable capacitor C1 and the second variable capacitor C2 are depicted with arrows indicating a variable nature of the first variable capacitor C1 and the second variable capacitor C2. The first variable capacitor C1 and the second variable capacitor C2 represent interdigitated capacitors whose capacitance values change as a function of a thickness of the sensing film. A fixed capacitor C is positioned in a center location between the first variable capacitor C1 and the second variable capacitor C2, representing a radiation capacitor formed by a gap in the split-ring resonator. The gap in the split-ring resonator can be referred to herein as a radiation gap of the inductive element.

The first variable capacitor C1 and the second variable capacitor C2 are connected in series with the fixed capacitor C and the inductor L, forming a resonant LC circuit. The electrode layer comprises a split-ring resonator coupled to at least one interdigitated capacitor, wherein the at least one interdigitated capacitor is connected in series with the split-ring resonator. The electrode structure forms a resonant circuit comprising an inductive element and a capacitive element, wherein the capacitive element comprises a first interdigitated capacitor and a second interdigitated capacitor positioned on opposite sides of a radiation gap of the inductive element.

This configuration allows a scattered field from the sensor system to have both a confined electric field near a sensor surface for thickness sensing, produced by the first variable capacitor C1 and the second variable capacitor C2, and a radiated field produced by the fixed capacitor C. The confined electric field region enables sensing of thickness changes near the sensor surface, while the radiation region can produce a backscattering signal for wireless readout. A resonance frequency of the circuit is controlled by capacitance values of the first variable capacitor C1 and the second variable capacitor C2, which change in response to degradation of the sensing film when exposed to an analyte of interest. Degradation of the sensing film alters a capacitance of the capacitive element to produce a detectable shift in a resonance frequency of the resonant circuit.

Referring to FIG. 2B, a detailed top view of the sensor design illustrates an arrangement of interdigitated capacitors within a split-ring resonator 205. The split-ring resonator 205 can be referred to herein as the electrode structure disposed on the biodegradable substrate. An upper portion of FIG. 2B displays an overall sensor structure with the split-ring resonator 205 having a dimension denoted as “a” and indicating positions of a first interdigitated capacitor 210A and a second interdigitated capacitor 210B. The first interdigitated capacitor 210A is positioned on a first side of the split-ring resonator 205, and the second interdigitated capacitor 210B is positioned on a second side of the split-ring resonator 205 opposite the first side. A cross-sectional reference line A-A′ is shown passing horizontally through the sensor structure.

With continued reference to FIG. 2B, a lower portion of the figure provides an enlarged view of the second interdigitated capacitor 210B, enclosed within a dashed boundary. This enlarged view reveals a finger-like electrode pattern characteristic of interdigitated capacitors, with multiple parallel electrode fingers extending from opposite sides and interleaving with one another. A dimension “B” indicates a width of an interdigitated region, and a cross-sectional reference line B-B′ passes vertically through the capacitor structure. The interdigitated capacitor design provides a confined electric field region near a sensor surface, enabling thickness sensing of a sensing film positioned above the electrodes. The first interdigitated capacitor 210A corresponds to the first variable capacitor C1 shown in FIG. 2A, and the second interdigitated capacitor 210B corresponds to the second variable capacitor C2 shown in FIG. 2A.

The split-ring resonator 205 can have a length dimension a of 10 mm. This dimension can be selected to fit within surgical tools while maintaining a relatively low resonance frequency suitable for the intended application. In some cases, the length dimension can be varied depending on specific surgical tool requirements and desired operating frequency ranges.

The first interdigitated capacitor 210A and the second interdigitated capacitor 210B can each comprise 8 capacitor arms (N=8). A gap width and an arm width can each be 0.2 mm, denoted as parameter s. This configuration can balance between a relatively high radar cross section (RCS) value and a detection range of approximately 60 μm. The spacing between adjacent electrode fingers and the number of finger pairs influence both a sensitivity of the sensor system and the RCS level, representing a trade-off between detection range and signal strength.

Additional dimensional parameters of the sensor design can include a gap dimension g of 1.8 mm, a dimension m of 1.76 mm, and a width dimension w of 1 mm. These parameters, along with the spacing parameter s of 0.2 mm, define the geometry of the split-ring resonator 205 and the first interdigitated capacitor 210A and the second interdigitated capacitor 210B. These dimensional values are exemplary, and other dimensional configurations can be employed depending on specific application requirements, desired operating frequency, and sensitivity characteristics.

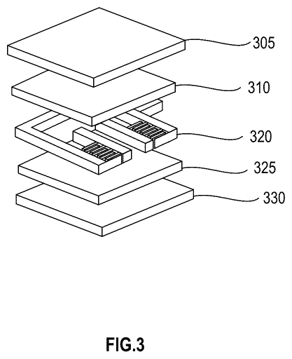

Referring to FIG. 3, an exploded view of a biocompatible sensor system 300 illustrates a layered construction of the sensor system. The biocompatible sensor system 300 includes several layers arranged in a stacked configuration. At a top of the assembly is a sensing film 305, which is configured to respond to an analyte of interest. Below the sensing film 305 is a passivation layer 310, which provides protection for underlying components. Molybdenum electrodes and SRR 320 are positioned beneath the passivation layer 310 and include interdigitated capacitor structures that form part of a resonant circuit. The molybdenum electrodes and SRR 320 can be referred to herein as an electrode layer disposed on a substrate. A substrate 325 is located below the molybdenum electrodes and SRR 320, providing structural support for the electrode layer. At a bottom of the assembly is a support layer 330, which serves as a base of the biocompatible sensor system 300. The support layer 330 can be referred to herein as a support layer disposed beneath the substrate.

With continued reference to FIG. 3, the support layer 330 comprises a polyimide material. In some cases, the support layer 330 comprises a 254 μm polyimide (e.g., Kapton) layer positioned beneath the substrate 325. The substrate 325 comprises polycaprolactone. In some cases, the substrate 325 comprises a 30 μm 35 w % v PCL substrate disposed on the support layer 330. This configuration provides a biocompatible sensor configuration, as opposed to a fully biodegradable configuration where the support layer 330 can be omitted.

The molybdenum electrodes and SRR 320 comprise molybdenum. In some cases, the molybdenum electrodes and SRR 320 have a thickness of 1 μm. The molybdenum electrodes and SRR 320 include the split-ring resonator 205, the first interdigitated capacitor 210A, and the second interdigitated capacitor 210B as described with reference to FIG. 2B. The electrode layer is disposed on the substrate 325.

The passivation layer 310 is disposed on the electrode layer. In some cases, the passivation layer 310 comprises a 1.6 μm 5 w % v PCL layer disposed between the molybdenum electrodes and SRR 320 and the sensing film 305. The passivation layer 310 provides electrical isolation and protection for the molybdenum electrodes and SRR 320 while allowing the electric field from the first interdigitated capacitor 210A and the second interdigitated capacitor 210B to extend into the sensing film 305.

The sensing film 305 is disposed on the passivation layer 310. The sensing film 305 comprises a pH-sensitive polymer configured to degrade in response to a pH level above a threshold value. The pH-sensitive polymer comprises a poly(meth)acrylate copolymer. In some cases, the poly(meth)acrylate copolymer comprises units of methacrylic acid and methyl methacrylate. The sensing film 305 can comprise various formulations depending on a target pH detection range.

In some cases, the sensing film 305 comprises a 50 μm 15 w % v ES100 Eudragit film that degrades at pH 7-8. In some cases, the sensing film 305 comprises a 48.5 μm 15 w % v EL100 Eudragit film that degrades at pH 6-7. In some cases, the sensing film 305 comprises a 28 μm ESL100 composite film that is a 1:1 blend of EL100 and ES100 and degrades at pH 6-8. The ESL100 composite film provides detection capability across a broader pH range by combining the degradation characteristics of both the EL100 and ES100 formulations. Other sensing films are also contemplated within the scope of the present disclosure, including, but not limited to, fibrinogen to detect blood, lipid coatings to detect pancreatic enzymes, a carbohydrate coating to detect amylase, and the like. Selection of a particular sensing film formulation can depend on a target pH range for detection in a specific clinical application.

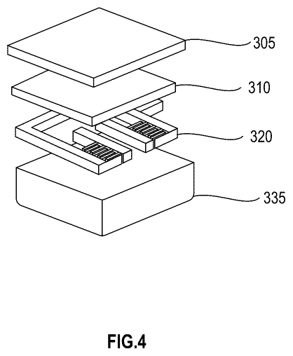

Referring to FIG. 4, an exploded view of a bioresorbable sensor system illustrates a layered construction of the sensor system. The bioresorbable sensor system includes several layers arranged in a stacked configuration. At a top of the assembly is the sensing film 305, which is configured to respond to an analyte of interest. Below the sensing film 305 is the passivation layer 310, which provides protection for underlying components. The molybdenum electrodes and SRR 320 are positioned beneath the passivation layer 310, forming active sensing elements of the bioresorbable sensor system. The molybdenum electrodes and SRR 320 include interdigitated capacitor structures that enable thickness sensing through changes in capacitance as the sensing film 305 degrades. At a bottom of the assembly is a substrate 335, which provides structural support for the bioresorbable sensor system.

With continued reference to FIG. 4, the bioresorbable sensor system differs from the biocompatible sensor system 300 described with reference to FIG. 3 in that the bioresorbable sensor system omits the support layer 330. The substrate 335 comprises polycaprolactone. In some cases, the substrate 335 comprises a 1-mm thick polycaprolactone substrate for full biodegradability. The biodegradable substrate can be referred to herein as the substrate 335. The increased thickness of the substrate 335 compared to the substrate 325 of the biocompatible sensor system 300 provides structural support without requiring a Kapton support layer beneath the substrate 335.

The substrate 335, the molybdenum electrodes and SRR 320, the passivation layer 310, and the sensing film 305 are each composed of bioresorbable materials. The electrode structure comprises molybdenum, which is a bioresorbable metal. The passivation layer 310 disposed over the electrode structure comprises polycaprolactone, which is a bioresorbable polymer. The sensing film 305 disposed over the passivation layer 310 is configured to degrade in response to a physiological parameter. The physiological parameter can comprise pH, and the sensing film 305 can comprise a pH-sensitive polymer that degrades at pH levels above a threshold value. The pH-sensitive polymer can comprise a poly(meth)acrylate copolymer comprising units of methacrylic acid and methyl methacrylate.

In some cases, the physiological parameter comprises enzymatic activity, and the sensing film 305 comprises a protein-based film that degrades in response to trypsin. The sensing film 305 can comprise a protein-based film configured to degrade in response to enzymatic activity. The protein-based film can comprise a gelatin-casein blend. In some cases, the sensing film 305 comprises a gelatin-casein blend with a 25:75 gelatin to casein ratio, which can be referred to as 75C25G. The 75C25G composition is optimized for trypsin detection. The gelatin-casein blend provides a bioresorbable sensing film that degrades when exposed to trypsin enzyme activity, enabling detection of enzymatic activity associated with anastomotic leakage in gastrointestinal surgery applications.

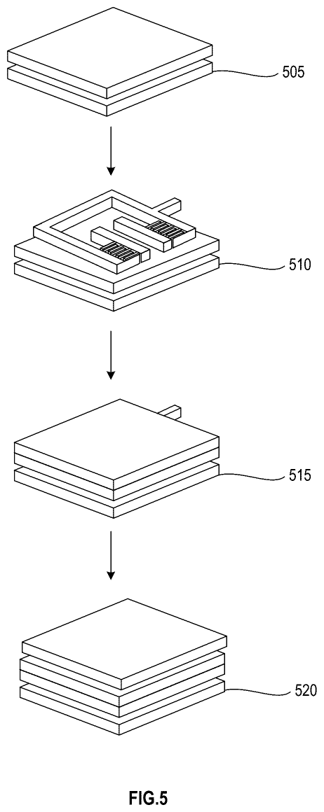

Referring to FIG. 5, a fabrication process flow for a biocompatible sensor is illustrated in four sequential steps. A method of fabricating a biodegradable sensor comprises providing a substrate, depositing an electrode layer on the substrate, depositing a passivation layer over the electrode layer, and depositing a sensing film over the passivation layer.

In a step 1 505, a substrate structure is shown comprising a light upper layer positioned on a dark lower layer. Providing the substrate comprises spin coating polycaprolactone onto the support layer 330. The fabrication process includes spin coating 4 layers of 35% w/v PCL on a polyimide wafer to form the substrate 325. The polyamide wafer serves as the support layer 330 and provides mechanical support during fabrication and handling of the biocompatible sensor system 300.

With continued reference to FIG. 5, in a step 2 510, the substrate from the step 1 505 has been processed to include patterned molybdenum electrodes and a split-ring resonator structure on an upper surface. The electrodes appear as a dark gray interdigitated pattern. Depositing an electrode layer on the substrate comprises depositing molybdenum and patterning the molybdenum to form the split-ring resonator 205 and the at least one interdigitated capacitor. The electrode layer comprises the split-ring resonator 205 and at least one interdigitated capacitor connected in series with the split-ring resonator 205. The at least one interdigitated capacitor can comprise the first interdigitated capacitor 210A and the second interdigitated capacitor 210B positioned on opposite sides of the split-ring resonator 205.

In a step 3 515, the passivation layer 310 has been deposited over the patterned electrodes. The passivation layer 310 is shown as a purple or lavender colored layer covering the substrate 325, with the underlying electrode structure visible beneath. Depositing the passivation layer 310 over the electrode layer comprises spin coating 5% w/v PCL. The fabrication process includes dicing the substrate 325 and spin coating 5% w/v PCL for the passivation layer 310. The passivation layer 310 provides electrical isolation and protection for the molybdenum electrodes and SRR 320.

In a step 4 520, the sensing film 305 has been applied as a topmost layer. The sensing film 305 appears as a teal or cyan colored coating that covers the passivation layer 310, completing a sensor stack structure. Depositing the sensing film 305 over the passivation layer 310 comprises depositing a pH-sensitive polymer comprising a poly(meth)acrylate copolymer comprising units of methacrylic acid and methyl methacrylate. The fabrication process includes spin coating 15% w/v Eudragit for the sensing film 305. The sensing film 305 is configured to change in thickness in response to an analyte of interest. The progression from the step 1 505 through the step 4 520 illustrates a sequential buildup of sensor layers on the substrate 325.

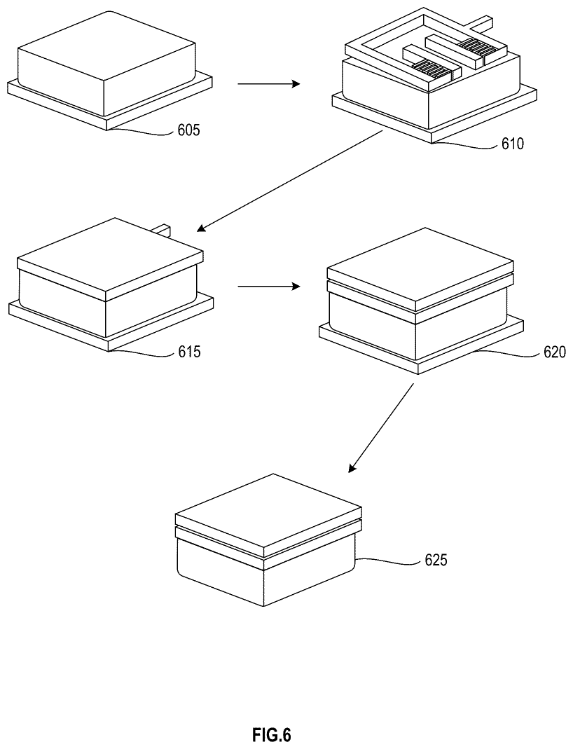

Referring to FIG. 6, a fabrication process flow for a bioresorbable sensor is illustrated in five sequential steps. The fabrication process for the bioresorbable sensor differs from the fabrication process for the biocompatible sensor described with reference to FIG. 5 in that the bioresorbable sensor fabrication process includes an alternative substrate preparation method and a step for removing the support layer to achieve full bioresorbability.

In a step 1 605, a substrate layer is prepared by melting polycaprolactone pellets between two polyamide sheets. The step 1 605 shows a hexagonal substrate layer with a gold or yellow colored bottom edge representing the polyimide sheet. The fabrication process includes melting PCL pellets onto a polyimide sheet as an alternative to spin coating for the substrate. This melting approach can produce a thicker substrate compared to the spin coating approach described with reference to FIG. 5, which can provide structural support for the bioresorbable sensor without requiring a permanent support layer. Pressing the kapton/PCL stack from above can create a very thin sheet of PCL. One of the Kapton sheets can then be removed providing a smooth surface where the electrodes can be patterned.

With continued reference to FIG. 6, in a step 2 610, the hexagonal structure includes patterned electrodes visible on a surface of the substrate, representing deposition and patterning of the molybdenum electrodes and SRR 320 and the split-ring resonator 205 structure. The step 2 610 corresponds to the step 2 510 described with reference to FIG. 5, where molybdenum is deposited and patterned to form the split-ring resonator 205, the first interdigitated capacitor 210A, and the second interdigitated capacitor 210B.

From the step 2 610, the fabrication process can proceed along alternative paths. A first path proceeds from the step 2 610 to a step 3 615, which shows the hexagonal structure with a lighter colored top layer added, representing addition of the passivation layer 310 or the sensing film 305. This first path corresponds to the fabrication sequence described with reference to FIG. 5 for producing a biocompatible sensor configuration.

A second path proceeds from the step 2 610 to a step 4 620, which depicts a hexagonal structure with multiple stacked layers including a cyan or light blue colored layer on top of a darker blue base layer. The step 4 620 represents an intermediate stage where the substrate with the electrode structure has been processed with additional layers. From the step 4 620, the fabrication process proceeds to a step 5 625, showing a final hexagonal structure with the cyan colored top layer, representing a completed sensor assembly with the sensing film 305 applied.

The parallel paths from the step 2 610 to either the step 3 615 or through the step 4 620 and the step 5 625 indicate alternative fabrication routes for producing either a biocompatible sensor configuration or a fully bioresorbable sensor configuration. For the fully bioresorbable sensor configuration, the fabrication process includes peeling off the polyimide sheet after fabrication to produce a fully bioresorbable sensor. The polyimide peel-off step removes the support layer 330 from the sensor assembly, leaving only bioresorbable materials including the substrate 335, the molybdenum electrodes and SRR 320, the passivation layer 310, and the sensing film 305. The resulting bioresorbable sensor can be implanted and can degrade within a body following use without requiring surgical removal.

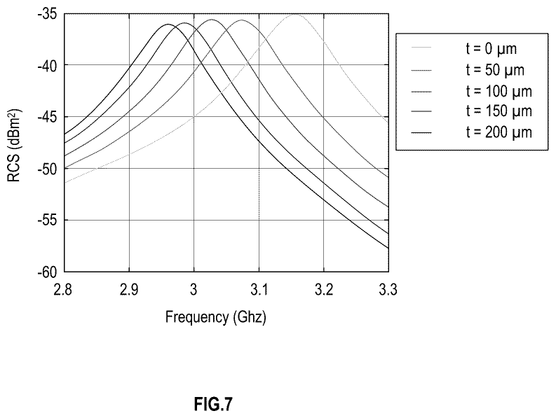

Referring to FIG. 7, a graph shows simulated radar cross section (RCS) in free space with different sensing film thicknesses. The horizontal axis represents frequency measured in gigahertz (GHz), ranging from approximately 2.8 GHz to 3.3 GHz. The vertical axis represents RCS measured in dBm squared, ranging from approximately −60 dBm squared to −35 dBm squared. The graph includes five curves corresponding to different sensing film thickness values denoted as parameter t.

With continued reference to FIG. 7, a first curve corresponds to t equals 0 micrometers, a second curve corresponds to t equals 50 micrometers, a third curve corresponds to t equals 100 micrometers, a fourth curve corresponds to t equals 150 micrometers, and a fifth curve corresponds to t equals 200 micrometers. Each curve exhibits a resonance peak at a particular frequency, with a peak RCS value being approximately −35 dBm squared for all curves. As the sensing film thickness increases from 0 micrometers to 200 micrometers, the resonance frequency shifts toward lower frequencies, demonstrating that the resonance frequency decreases as the thickness of the sensing film increases.

The curve for t equals 0 micrometers shows a resonance peak at approximately 3.16 GHz, while the curve for t equals 200 micrometers shows a resonance peak at approximately 2.9 GHz. The resonance frequency shifts from 3.16 GHz to 3.03 GHz as the sensing film thickness increases from 0 micrometers to 100 micrometers. A sensitivity of the sensor system can be calculated based on the simulated data. The sensitivity is defined as a frequency percentage shift per a given change in sensing film thickness. For a 100 micrometer thickness change, the sensor system achieves a sensitivity of 4.2% in free space. This sensitivity metric quantifies the frequency shift response of the sensor system to changes in the sensing film thickness.

The sensing film thickness can be in a range of 30-50 micrometers for detectable degradation while maintaining reasonable degradation time. Sensors measured in experimental configurations have thicknesses in the 30-50 micrometer range, which is detectable by the sensor system while providing a degradation time suitable for postoperative monitoring applications. The simulated RCS results demonstrate that the sensor system can resolve thickness changes within this range based on the resonance frequency shift characteristics shown in FIG. 7. While a sensing film thickness of 30-50 micrometers is discussed above, the disclosure is not so limited. Rather, as those skilled in the art would understand, other thicknesses are contemplated by the present disclosure.

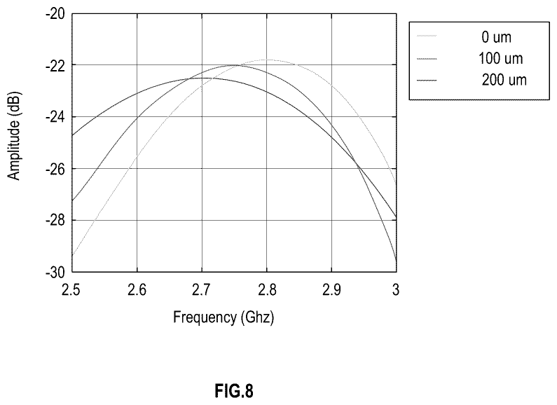

Referring to FIG. 8, a graph shows simulated radar cross section (RCS) amplitude in decibels (dB) as a function of frequency in gigahertz (GHz) for different sensing film thicknesses in a phantom environment. The phantom environment simulates human tissue with a permittivity (εᵣ) of 50. The vertical axis represents amplitude ranging from approximately −30 dB to −20 dB, while the horizontal axis represents frequency ranging from 2.5 GHz to 3 GHz. Three curves are displayed corresponding to sensing film thicknesses of 0 μm, 100 μm, and 200 μm.

With continued reference to FIG. 8, the curve corresponding to 0 μm sensing film thickness exhibits a peak amplitude of approximately −22 dB at a frequency of approximately 2.8 GHz. The curve corresponding to 100 μm sensing film thickness shows a peak amplitude of approximately −22 dB at a frequency of approximately 2.75 GHz, representing a shift toward lower frequencies compared to the 0 μm case. The curve corresponding to 200 μm sensing film thickness displays a peak amplitude of approximately −22 dB at a frequency of approximately 2.7 GHz, demonstrating a further shift toward lower frequencies. The graph illustrates that as the sensing film thickness increases, the resonance frequency shifts to lower values while the peak amplitude remains relatively constant across all three thickness conditions.

The resonance frequency shifts from 2.8 GHz to 2.75 GHz as the sensing film thickness increases from 0 μm to 100 μm in the phantom environment. The sensor system achieves a sensitivity of 1.8% in a phantom environment per 100 μm thickness change. This sensitivity in the phantom environment is lower than the 4.2% sensitivity achieved in free space as described with reference to FIG. 7. The reduced sensitivity in the phantom environment results from the high permittivity contrast between the polycaprolactone substrate (εᵣ=3.5) and the surrounding human tissue (εᵣ=50), which causes the scattered electric fields from the interdigitated capacitors to be confined within the sensing film rather than penetrating into the phantom region.

The sensor system can resolve sensing film thickness changes of 6.2 μm. This thickness resolution is determined based on a frequency step of 3.125 MHz used in measurement configurations. Given that the resonance frequency shifts from 2.8 GHz to 2.75 GHz (a shift of 50 MHz) as the sensing film thickness increases from 0 μm to 100 μm, each 3.125 MHz frequency shift corresponds to a thickness change of approximately 6.2 μm. This resolution enables detection of small changes in the sensing film thickness as the sensing film degrades in response to an analyte of interest, providing sufficient sensitivity for monitoring physiological parameters in postoperative applications.

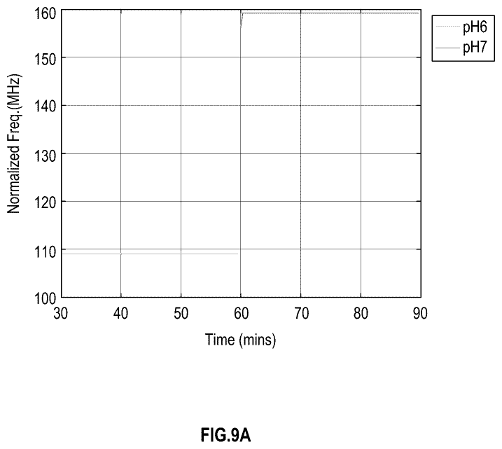

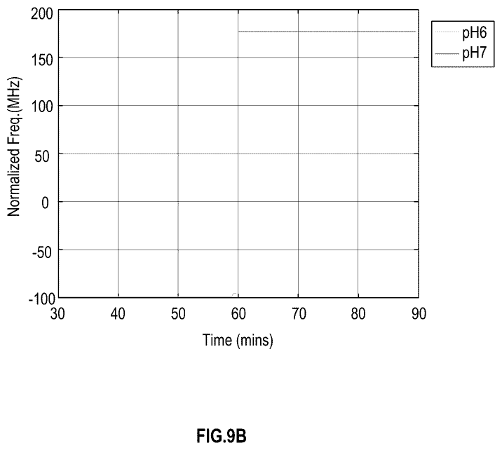

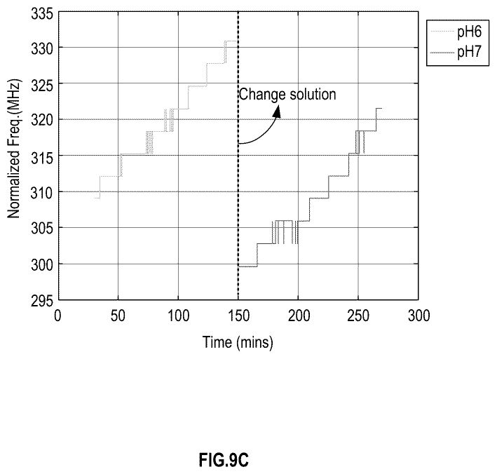

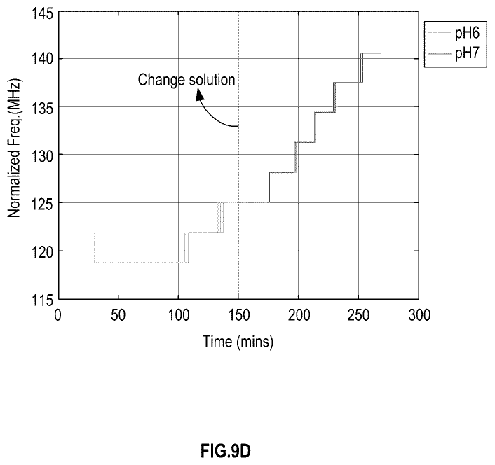

Referring to FIG. 9A, a graph shows normalized frequency in MHz as a function of time in minutes for two different pH conditions. The vertical axis represents normalized frequency measured in MHz, ranging from 100 to 160 MHz. The horizontal axis represents time measured in minutes, ranging from 30 to 90 minutes. Two data series are plotted on the graph, with a first series corresponding to pH 6 shown as a horizontal line at approximately 110 MHz that remains stable from 30 minutes to approximately 60 minutes. A second series corresponding to pH 7 is shown as a horizontal line at approximately 159 MHz that begins at approximately 60 minutes and continues to 90 minutes. The graph demonstrates that the sensor system exhibits distinct resonance frequency responses at different pH levels, with the pH 7 condition producing a higher normalized frequency compared to the pH 6 condition.

With continued reference to FIG. 9A, the sensor system is configured for wired measurement using a 50 Ω coplanar waveguide (CPW) feed on a testing PCB. The testing PCB provides a controlled excitation method for characterizing sensor performance before conducting wireless measurements. The sensor system operates at a temperature of 37° C. to mimic human body conditions. A temperature chamber maintains the 37° C. operating temperature during wired measurements to simulate the thermal environment that the sensor system would experience when implanted in a patient.

Referring to FIG. 9B, a graph shows normalized frequency in MHz as a function of time in minutes for two different pH conditions. The vertical axis represents normalized frequency measured in MHz, ranging from approximately −100 MHz to 200 MHz. The horizontal axis represents time measured in minutes, spanning from 30 minutes to 90 minutes. Two data series are plotted on the graph, with a first series corresponding to pH 6 shown as a horizontal line at approximately −100 MHz that remains stable from 30 minutes to approximately 60 minutes. A second series corresponding to pH 7 is shown as a horizontal line at approximately 175 MHz that begins at approximately 60 minutes and continues to 90 minutes. The graph demonstrates that the sensor system exhibits distinct resonance frequency responses at different pH levels, with the pH 7 condition producing a higher normalized frequency compared to the pH 6 condition.

Referring to FIG. 9C, a graph shows normalized frequency in MHz as a function of time in minutes for two different pH conditions. The vertical axis represents normalized frequency measured in MHz, ranging from approximately 295 MHz to 335 MHz. The horizontal axis represents time measured in minutes, ranging from 0 to 300 minutes. A first trace corresponding to pH 6 is shown, and a second trace corresponding to pH 7 is shown. A vertical dashed line at approximately 150 minutes indicates a point where the solution is changed, as denoted by an annotation labeled “Change solution” with arrows pointing to both traces at this transition point.

With continued reference to FIG. 9C, the pH 6 trace begins at approximately 309 MHz at time zero and exhibits a stepwise increase over time, reaching approximately 331 MHz before the solution change, after which the trace is no longer present. The pH 7 trace begins at approximately 300 MHz at the solution change point and exhibits a stepwise increase over time, reaching approximately 322 MHz by 300 minutes. Both traces display a characteristic staircase pattern indicating discrete frequency shifts occurring at various time intervals throughout the measurement period. The stepwise frequency shifts correspond to degradation of the sensing film as the sensing film responds to the elevated pH conditions.

Referring to FIG. 9D, a graph shows normalized frequency in MHz as a function of time in minutes for two different pH conditions. The vertical axis represents normalized frequency measured in MHz, ranging from approximately 115 MHz to 145 MHz. The horizontal axis represents time measured in minutes, ranging from 0 to 300 minutes. A first trace corresponding to pH 6 is shown, and a second trace corresponding to pH 7 is shown. A vertical dashed line at approximately 150 minutes indicates a point where the solution is changed, as denoted by an annotation labeled “Change solution” with an arrow pointing to the dashed line.

With continued reference to FIG. 9D, the pH 6 trace begins at approximately 120 MHz and shows a stepwise increase over time, reaching approximately 128 MHz before the solution change, after which the trace terminates. The pH 7 trace begins at approximately 128 MHz at the time of the solution change and continues with a stepwise increase, reaching approximately 140 MHz by the end of the measurement period at 300 minutes. Both traces exhibit a characteristic staircase pattern indicating discrete frequency shifts over time in response to the respective pH conditions.

The sensor system achieves response times under 20 minutes for pH detection. The response time represents a duration from initial exposure to a pH condition until a detectable frequency shift is observed. The rapid response time enables timely detection of pH changes that can indicate anastomotic leakage in postoperative monitoring applications. The sensor system achieves quality factors of 6.5±1.8 at pH 6 and 9.7±2.3 at pH 7. The quality factor is determined from background readings using a formula where the resonance frequency is divided by a full width at half maximum of the resonance peak. The quality factor values indicate the sharpness of the resonance response, with higher quality factors corresponding to more distinct resonance peaks that facilitate accurate frequency shift detection.

The wireless measurement uses a scanning frequency range of 1-6 GHz to achieve spatial resolution of 3 cm in free space. The wide frequency band enables range resolution sufficient to distinguish the sensor response from background reflections and clutter in the measurement environment. A horn antenna phase center can be placed approximately 30 cm away from the sensor system for wireless interrogation. The wireless measurement uses time gating and background subtraction post-processing techniques to increase signal-to-noise ratio. Time gating isolates the sensor response in the time domain by selecting a time window corresponding to the sensor location, while background subtraction removes reflections from the measurement environment by subtracting a reference measurement taken without the sensor present. These post-processing techniques enable extraction of the sensor resonance frequency from the wireless measurement data with sufficient accuracy for detecting thickness changes in the sensing film.

The sensor system can include a two-tag configuration with a reference tag operating on an orthogonal polarization channel to calibrate for uncertain operating frequency when implanted. When the sensor system is implanted within a body, the operating frequency of the sensor system can be uncertain due to environmental and inter-subject variabilities and frequency dependent tissue permittivity. The permittivity of surrounding materials affects the capacitance values of the interdigitated capacitors, which in turn affects the resonance frequency of the sensor system. Because the environment within a body can vary between patients and between different implantation locations, the operating frequency of the sensor system when implanted can differ from the operating frequency measured in a laboratory environment.

The two-tag configuration addresses this uncertainty by providing a baseline operating frequency reference. The reference tag is placed adjacent to the sensor and operates on an orthogonal channel relative to the sensor. The sensor system operates on a first channel, referred to as channel X, where an x-polarized electromagnetic wave is transmitted and received. The reference tag operates on a second channel, referred to as channel Y, where a y-polarized electromagnetic wave is transmitted and received. Because the two channels use orthogonal polarizations, the sensor and the reference tag are excited separately without interference between the two tags.

The reference tag can have a fixed sensing film thickness that does not change in response to the analyte of interest. In some cases, a pH-insensitive film is placed on the reference tag. The pH-insensitive film can comprise polycaprolactone or another material that does not degrade in response to pH changes within the detection range of the sensor system. Because the reference tag has a fixed sensing film thickness, the resonance frequency of the reference tag does not change in response to the analyte of interest. The resonance frequency of the reference tag provides a baseline operating frequency that reflects the effects of the implantation environment on the resonance characteristics of the sensor system.

Sensing information is extracted by observing a peak frequency difference between the two channels. When the sensor system is interrogated wirelessly, the resonance frequency of the sensor in channel X and the resonance frequency of the reference tag in channel Y are both measured. As the sensing film on the sensor degrades in response to the analyte of interest, the resonance frequency of the sensor in channel X shifts while the resonance frequency of the reference tag in channel Y remains constant. The change in the sensing film thickness can be determined by calculating the difference between the resonance frequency of the sensor and the resonance frequency of the reference tag.

The two-tag configuration compensates for environmental effects that would otherwise introduce uncertainty into the measurement. Both the sensor and the reference tag experience the same environmental conditions, including the permittivity of surrounding tissue and temperature variations. Because both tags are affected equally by these environmental factors, the peak frequency difference between the two channels isolates the frequency shift caused by degradation of the sensing film from frequency shifts caused by environmental variations. This calibration approach enables accurate detection of sensing film thickness changes even when the absolute operating frequency of the sensor system is uncertain due to the implantation environment.

The sensor system is configured for wireless interrogation using a monostatic measurement system. The monostatic measurement system comprises a dual-ridge horn antenna that transmits electromagnetic waves toward the sensor system and receives backscattered signals from the sensor system. The dual-ridge horn antenna provides wideband operation across the frequency range used for interrogating the sensor system. The sensor system is configured for wireless interrogation at a reading distance of 30 cm from a horn antenna phase center. This reading distance provides sufficient separation between the antenna and the sensor system for practical clinical applications while maintaining adequate signal strength for detecting resonance frequency shifts in the sensor system.

The sensor system can be used with a bio-matched antenna to improve wireless interrogation performance when the sensor system is implanted within a body. The bio-matched antenna can incorporate a matching layer positioned between the antenna and the body surface. The matching layer reduces reflections at the interface between air and human tissue by providing an intermediate permittivity transition. The matching layer can comprise a dielectric material with a permittivity value between the permittivity of air and the permittivity of human tissue. By reducing interface reflections, the matching layer increases the amount of electromagnetic energy that penetrates into the body to reach the sensor system and increases the amount of backscattered energy that exits the body to reach the receiving antenna.

In some cases, the bio-matched antenna can incorporate a focusing lens. The focusing lens concentrates electromagnetic energy toward the implanted sensor system, increasing the signal strength at the sensor location. The focusing lens can comprise a tapered graded index lens or another lens configuration that provides enhanced penetration for near-field imaging applications. The focusing lens can improve the signal-to-noise ratio of the wireless interrogation by directing more electromagnetic energy toward the sensor system and collecting more of the backscattered signal from the sensor system.

In some cases, the bio-matched antenna can incorporate an imaging array. The imaging array comprises multiple antenna elements arranged in a spatial configuration. The imaging array can provide spatial selectivity for locating the sensor system within the body and can improve signal quality by combining signals from multiple antenna elements. The imaging array can be configured for microwave imaging applications where the sensor system location within the body is determined based on the spatial characteristics of the received signals.

The sensor system can include an additional isolation layer to confine electric field lines within the device when operating in conductive environments. When the sensor system is implanted in tissue or surrounded by conductive fluids, the conductivity of the surrounding environment can reduce the quality factor of the resonant circuit by providing a path for energy dissipation. The additional isolation layer can be positioned between the electrode structure and the surrounding conductive environment. The additional isolation layer can comprise a dielectric material that prevents direct contact between the electrode structure and the conductive environment. By confining the electric field lines within the device, the additional isolation layer maintains the quality factor of the resonant circuit and preserves the sensitivity of the sensor system for detecting changes in the sensing film thickness.

The sensing film can be configured for additive sensing where a material builds up on a sensor surface. In the additive sensing configuration, the sensing film thickness increases over time as material accumulates on the sensor surface, in contrast to the subtractive sensing configuration where the sensing film degrades and decreases in thickness. The additive sensing configuration can be used for detecting bleeding by monitoring blood clot formation on the sensor surface. When bleeding occurs near the sensor system, blood can contact the sensor surface and form a clot. As the blood clot forms and grows on the sensor surface, the effective thickness of material above the interdigitated capacitors increases, which changes the capacitance values and produces a detectable shift in the resonance frequency of the sensor system. The additive sensing configuration enables detection of bleeding events in postoperative monitoring applications.

The sensor system can be configured for placement in the Pouch of Morrison for detecting leakage from pancreatic, liver, or colorectal surgeries. The Pouch of Morrison, also referred to as the hepatorenal recess, is an anatomical space located between the liver and the right kidney. The Pouch of Morrison represents a dependent location within the abdominal cavity where fluids can accumulate following surgical procedures. Placement of the sensor system in the Pouch of Morrison enables detection of fluid accumulation that can indicate leakage from nearby surgical sites. The sensor system can detect changes in pH or enzymatic activity of fluids that accumulate in the Pouch of Morrison, providing an indication of anastomotic leakage or other postoperative complications. The wireless interrogation capability of the sensor system enables monitoring of the Pouch of Morrison location without requiring direct access to the implantation site following surgery. In other embodiments, the sensor can be configured for placement in other locations within the abdomen, which may be of concern.

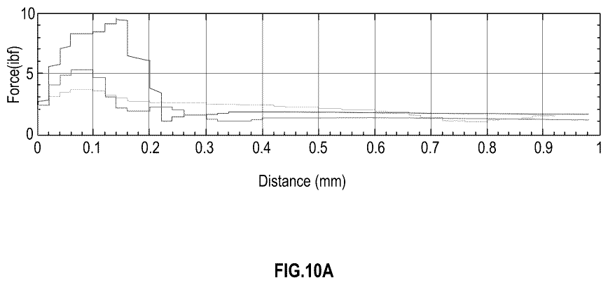

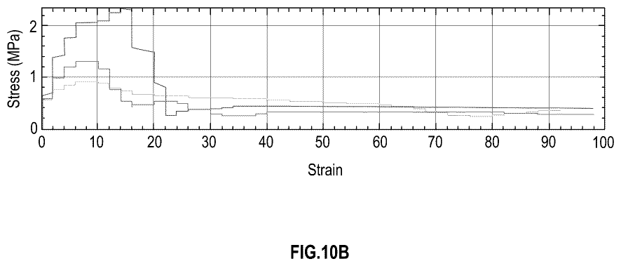

Referring to FIG. 10A, a graph shows force in pounds-force (lbf) as a function of distance in millimeters (mm) for mechanical testing of sensing films. The vertical axis represents force measured in lbf, ranging from 0 to 10, while the horizontal axis represents distance measured in mm, ranging from 0 to 1. Three curves are displayed on the graph, each representing a separate test sample of the sensing film material. Each curve shows a characteristic force-distance relationship where the force remains relatively low and stable at distances below approximately 0.2 mm, then increases as the distance increases from approximately 0.2 mm to 0.6 mm, and finally plateaus or slightly decreases at distances above approximately 0.6 mm.

With continued reference to FIG. 10A, one curve exhibits the highest peak force values, reaching approximately 8 to 9 lbf, while the other curves show lower peak force values in the range of approximately 5 to 7 lbf. The three curves demonstrate similar overall trends but with variations in their specific force magnitudes and transition characteristics, indicating differences in mechanical properties among the tested samples. The force-distance curves provide data for evaluating the mechanical behavior of the sensing film materials under tensile loading conditions. The initial low-force region corresponds to slack removal and initial engagement of the sample, while the increasing force region corresponds to elastic deformation of the sensing film material. The plateau or decrease in force at higher distances corresponds to yielding or failure of the sensing film material.

Referring to FIG. 10B, a graph shows stress-strain curves for mechanical testing of film samples. The horizontal axis represents strain with values ranging from 0 to 100, while the vertical axis represents stress measured in megapascals (MPa) with values ranging from 0 to approximately 2.5 MPa. Three curves are displayed on the graph, each demonstrating the mechanical behavior of a film sample under tensile loading conditions. The curves show an initial region of relatively low stress at low strain values, followed by a region where stress increases more rapidly with increasing strain. The curves exhibit variations in their stress-strain relationships, with some samples showing higher ultimate stress values than others. Two curves extend to higher strain values approaching 100, while a third curve terminates at a lower strain value.

With continued reference to FIG. 10B, the stress-strain curves illustrate the tensile strength and elongation characteristics of the tested film materials. The sensing film samples can achieve an elastic modulus of approximately 2.01 MPa and a tensile strength of approximately 2.24 MPa for 28-day-old films. Freshly prepared films (0-day-old) can demonstrate an elastic modulus of approximately 1.44 MPa and a tensile strength of approximately 2.13 MPa. The 28-day-old films exhibit enhanced stiffness and strength compared to the freshly prepared films, indicating a potential increase in crosslinking or structural stability in the films with aging.

The mechanical testing results demonstrate stability of the sensing film materials over time. The comparison between 0-day-old and 28-day-old films shows that the mechanical properties of the sensing films are maintained or improved following a 28-day storage period. The elastic modulus increases from approximately 1.44 MPa to approximately 2.01 MPa over the 28-day period, representing an increase in stiffness. The tensile strength increases from approximately 2.13 MPa to approximately 2.24 MPa over the same period, representing an increase in strength. These results suggest that the sensing film materials do not degrade mechanically during storage and can maintain structural integrity for implantable sensor applications.

The mechanical properties of the sensing films demonstrate suitability for implantable applications. The elastic modulus values in the range of 1.4 to 2.0 MPa indicate that the sensing films have moderate elasticity that can accommodate mechanical stresses encountered in an implanted environment. The tensile strength values in the range of 2.1 to 2.2 MPa indicate that the sensing films can withstand tensile forces without failure during handling, implantation, and operation of the sensor system. The mechanical stability over time indicates that the sensing films can maintain their structural properties during the postoperative monitoring period, which can extend for several days to weeks following surgery. The combination of adequate mechanical strength and stability over time supports the use of the sensing film materials in biodegradable sensor systems for postoperative monitoring applications.

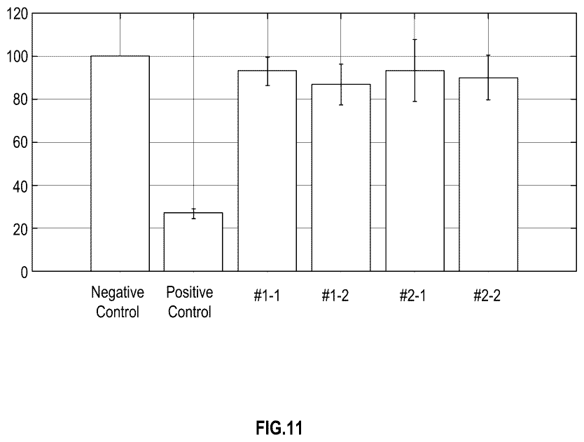

Referring to FIG. 11, a bar chart illustrates cell viability results from cytotoxicity testing of sensor film samples compared to control groups. The vertical axis represents a percentage scale ranging from 0 to 120, while the horizontal axis displays different sample categories. A negative control bar extends to approximately 100 percent, representing baseline cell viability where cells are cultured in standard medium without exposure to any materials. A positive control bar shows significantly reduced cell viability at approximately 27 percent, representing cells exposed to a known cytotoxic agent (1% SDS). The positive control validates the sensitivity of the cytotoxicity assay by demonstrating that the assay can detect cytotoxic effects when present.

With continued reference to FIG. 11, four test sample bars are displayed, labeled as 1-1, 1-2, 2-1, and 2-2. Sample 1 represents 28-day-old 75C25G films, while sample 2 corresponds to 0-day-old 75C25G films. Each test sample bar extends to approximately 85 to 95 percent cell viability. Error bars are present on each of the test sample bars, indicating measurement variability across replicate samples. The test samples demonstrate cell viability levels substantially higher than the positive control and approaching the negative control baseline, indicating non-cytotoxic characteristics of the tested sensor film materials.

The cytotoxicity testing follows an MTS assay protocol where L929 fibroblast cells are exposed to sample extract medium for 24 hours. The sample extract medium is prepared by immersing 1.0×1.0 cm² film samples in complete cell culture medium at 37° C. for 12 hours. For the assay, 100 μL of L929 cell suspension at a density of 1.0×10⁵ cells/mL is seeded into 96-well plates and incubated for 24 hours to allow cell adhesion. The culture medium is then replaced with sample extract media derived from the sensor film samples. Cells are incubated for an additional 24 hours, followed by addition of 20 μL of MTS reagent to each well. The reaction proceeds for 1 hour, after which optical density is measured at 490 nm using a plate reader. Each experimental condition is performed in six replicates, and relative cell viability is calculated as a percentage of the negative control group.

The 28-day-old films yield average cell viabilities of 92.81%±6.61 and 86.66%±9.47 across two replicates. The freshly prepared (0-day-old) films demonstrate average viabilities of 93.11%±14.27 and 89.88%±10.17 across replicates. Both the 28-day-old films and the 0-day-old films surpass a 70% cell viability threshold specified by ISO 10993-5, confirming non-cytotoxic characteristics of the sensor film materials. The ISO 10993-5 standard provides guidelines for in vitro cytotoxicity testing of medical devices, and materials that maintain cell viability above 70% are considered non-cytotoxic according to this standard.

Slight variability in cell viability is observed across replicates, particularly for the freshly prepared films, which exhibit higher standard deviations compared to the 28-day-old films. The higher variability in the freshly prepared films can result from minor inconsistencies in sample preparation or localized variations in material properties. The 28-day-old films demonstrate more consistent cell viability results, suggesting that the aging process can improve uniformity of the film properties. The cytotoxicity results demonstrate that the sensor film materials maintain biocompatibility over time, with both freshly prepared and 28-day-old films exhibiting non-cytotoxic characteristics suitable for implantable sensor applications.

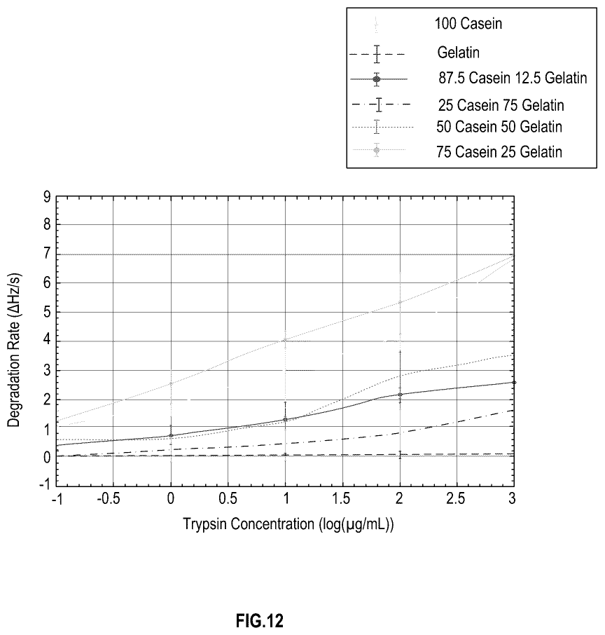

Referring to FIG. 12, a graph shows degradation rate as a function of trypsin concentration for various protein blend compositions. The vertical axis represents degradation rate measured in delta Hz per second, ranging from negative one to nine. The horizontal axis represents trypsin concentration on a logarithmic scale measured in log of micrograms per milliliter, ranging from negative one to three. The graph displays six different curves corresponding to different gelatin to casein ratios in the protein blend films.

With continued reference to FIG. 12, a first curve represents a composition of one hundred percent casein. A second curve represents a composition of eighty-seven point five percent casein and twelve point five percent gelatin. A third curve represents a composition of seventy-five percent casein and twenty-five percent gelatin. A fourth curve, shown as a dashed line, represents a composition of fifty percent casein and fifty percent gelatin. A fifth curve represents a composition of twenty-five percent casein and seventy-five percent gelatin. A sixth curve represents a composition of one hundred percent gelatin. Error bars are displayed on several data points indicating measurement variability.

The curves demonstrate that compositions with higher casein content generally exhibit higher degradation rates at elevated trypsin concentrations. The one hundred percent casein composition and the seventy-five percent casein with twenty-five percent gelatin composition show the highest degradation rates, reaching approximately seven delta Hz per second at the highest trypsin concentration tested. The one hundred percent gelatin composition shows minimal degradation rate response across all trypsin concentrations, remaining near zero throughout the tested range.

The seventy-five percent casein and twenty-five percent gelatin blend, referred to as 75C25G, exhibits superior trypsin detection performance compared to other blend compositions. The 75C25G composition demonstrates a high initial reaction rate while maintaining sensitivity across a broad range of trypsin concentrations. The 75C25G composition achieves a balance between the high degradation rate characteristic of casein-rich compositions and the structural properties provided by gelatin content. The sensing film for trypsin detection achieves a limit of detection of 7.81×10⁻¹¹ M. This limit of detection represents a 10-fold improvement in trypsin sensitivity compared to pure gelatin films and a 5-fold enhancement over pure casein films.

The gelatin-casein sensing film has a thickness of approximately 250 nm when deposited by spin coating at 5000 rpm for 30 seconds. The spin coating parameters control the thickness of the deposited sensing film, with higher spin speeds producing thinner films. The 250 nm film thickness provides a sensing layer that is sufficiently thin to enable rapid degradation in response to trypsin activity while maintaining adequate coverage over the electrode structure for capacitance-based sensing.

The molybdenum electrode layer has a conductivity of σ=1.76×10⁷ S/m. The conductivity of the molybdenum electrode layer affects the quality factor of the resonant circuit and the radar cross section level of the sensor system. The molybdenum conductivity is lower than the conductivity of copper or gold, which can result in higher resistive losses in the electrode structure. The trade-off between biodegradability and conductivity is addressed by selecting electrode geometry parameters that maintain adequate signal strength for wireless interrogation while using the bioresorbable molybdenum material.

The disclosed technology can be further understood according to the following clauses:
Clause 1: A sensor system, comprising: a substrate; an electrode layer disposed on the substrate, the electrode layer comprising a split-ring resonator coupled to at least one interdigitated capacitor, wherein the at least one interdigitated capacitor is connected in series with the split-ring resonator; a passivation layer disposed on the electrode layer; and a sensing film disposed on the passivation layer, wherein the sensing film is configured to change in thickness in response to an analyte of interest, and wherein a change in thickness of the sensing film causes a shift in a resonance frequency of the sensor system.
Clause 2: The sensor system of clause 1, wherein the substrate comprises a mechanically stable biodegradable polymer (e.g., polycaprolactone).
Clause 3: The sensor system of clause 1, wherein the electrode layer comprises a biodegradable metal (e.g., molybdenum).
Clause 4: The sensor system of clause 1, wherein the sensing film comprises a pH-sensitive polymer configured to degrade in response to a pH level above a threshold value.
Clause 5: The sensor system of clause 4, wherein the pH-sensitive polymer comprises a poly(meth)acrylate copolymer.
Clause 6: The sensor system of clause 1, wherein the sensing film comprises a protein-based film configured to degrade in response to enzymatic activity.
Clause 7: The sensor system of clause 6, wherein the protein-based film comprises a gelatin-casein blend.
Clause 8: The sensor system of clause 1, wherein the at least one interdigitated capacitor comprises: a first interdigitated capacitor positioned on a first side of the split-ring resonator; and a second interdigitated capacitor positioned on a second side of the split-ring resonator opposite the first side.
Clause 9: The sensor system of clause 1, further comprising a support layer disposed beneath the substrate, wherein the support layer comprises a polyimide.
Clause 10: The sensor system of clause 1, wherein the substrate, the electrode layer, the passivation layer, and the sensing film are each composed of bioresorbable materials.
Clause 11: A sensor system for postoperative monitoring, comprising: a biodegradable substrate; an electrode structure disposed on the biodegradable substrate, the electrode structure forming a resonant circuit comprising an inductive element and a capacitive element, wherein the capacitive element comprises a first interdigitated capacitor and a second interdigitated capacitor positioned on opposite sides of a radiation gap of the inductive element; a passivation layer disposed over the electrode structure; and a sensing film disposed over the passivation layer, wherein the sensing film is configured to degrade in response to a physiological parameter, and wherein degradation of the sensing film alters a capacitance of the capacitive element to produce a detectable shift in a resonance frequency of the resonant circuit.
Clause 12: The sensor system of clause 11, wherein the biodegradable substrate comprises polycaprolactone.
Clause 13: The sensor system of clause 11, wherein the electrode structure comprises molybdenum.
Clause 14: The sensor system of clause 11, wherein the physiological parameter comprises pH, and wherein the sensing film comprises a pH-sensitive polymer that degrades at pH levels above a threshold value.
Clause 15: The sensor system of clause 14, wherein the pH-sensitive polymer comprises a poly(meth)acrylate copolymer comprising units of methacrylic acid and methyl methacrylate.
Clause 16: The sensor system of clause 11, wherein the physiological parameter comprises enzymatic activity, and wherein the sensing film comprises a protein-based film that degrades in response to trypsin.
Clause 17: A method of fabricating a biodegradable sensor, comprising: providing a substrate; depositing an electrode layer on the substrate, wherein the electrode layer comprises a split-ring resonator and at least one interdigitated capacitor connected in series with the split-ring resonator; depositing a passivation layer over the electrode layer; and depositing a sensing film over the passivation layer, wherein the sensing film is configured to change in thickness in response to an analyte of interest.
Clause 18: The method of clause 17, wherein providing the substrate comprises spin coating polycaprolactone onto a support layer.
Clause 19: The method of clause 18, wherein depositing the electrode layer comprises depositing molybdenum and patterning the molybdenum to form the split-ring resonator and the at least one interdigitated capacitor.
Clause 20: The method of clause 17, wherein depositing the sensing film comprises depositing a pH-sensitive polymer comprising a poly(meth)acrylate copolymer comprising units of methacrylic acid and methyl methacrylate.

A number of implementations have been described. Nevertheless, it will be understood that various modifications may be made without departing from the spirit and scope of the disclosure. Accordingly, other implementations are within the scope of the following claims.

1. A sensor system, comprising:
a substrate;
an electrode layer disposed on the substrate, the electrode layer comprising a split-ring resonator coupled to at least one interdigitated capacitor, wherein the at least one interdigitated capacitor is connected in series with the split-ring resonator;
a passivation layer disposed on the electrode layer; and
a sensing film disposed on the passivation layer, wherein the sensing film is configured to change in thickness in response to an analyte of interest, and wherein a change in thickness of the sensing film causes a shift in a resonance frequency of the sensor system.

2. The sensor system of claim 1, wherein the substrate comprises a mechanically stable biodegradable polymer.

3. The sensor system of claim 1, wherein the electrode layer comprises a biodegradable metal.

4. The sensor system of claim 1, wherein the sensing film comprises a pH-sensitive polymer configured to degrade in response to a pH level above a threshold value.

5. The sensor system of claim 4, wherein the pH-sensitive polymer comprises a poly(meth)acrylate copolymer.

6. The sensor system of claim 1, wherein the sensing film comprises a protein-based film configured to degrade in response to enzymatic activity.

7. The sensor system of claim 6, wherein the protein-based film comprises a gelatin-casein blend.

8. The sensor system of claim 1, wherein the at least one interdigitated capacitor comprises:
a first interdigitated capacitor positioned on a first side of the split-ring resonator; and
a second interdigitated capacitor positioned on a second side of the split-ring resonator opposite the first side.

9. The sensor system of claim 1, further comprising a support layer disposed beneath the substrate, wherein the support layer comprises a polyimide.

10. The sensor system of claim 1, wherein the substrate, the electrode layer, the passivation layer, and the sensing film are each composed of bioresorbable materials.

11. A sensor system for postoperative monitoring, comprising:
a biodegradable substrate;
an electrode structure disposed on the biodegradable substrate, the electrode structure forming a resonant circuit comprising an inductive element and a capacitive element, wherein the capacitive element comprises a first interdigitated capacitor and a second interdigitated capacitor positioned on opposite sides of a radiation gap of the inductive element;
a passivation layer disposed over the electrode structure; and
a sensing film disposed over the passivation layer, wherein the sensing film is configured to degrade in response to a physiological parameter, and wherein degradation of the sensing film alters a capacitance of the capacitive element to produce a detectable shift in a resonance frequency of the resonant circuit.

12. The sensor system of claim 11, wherein the biodegradable substrate comprises polycaprolactone.

13. The sensor system of claim 11, wherein the electrode structure comprises a biodegradable metal.

14. The sensor system of claim 11, wherein the physiological parameter comprises pH, and wherein the sensing film comprises a pH-sensitive polymer that degrades at pH levels above a threshold value.

15. The sensor system of claim 14, wherein the pH-sensitive polymer comprises a poly(meth)acrylate copolymer comprising units of methacrylic acid and methyl methacrylate.

16. The sensor system of claim 11, wherein the physiological parameter comprises enzymatic activity, and wherein the sensing film comprises a protein-based film that degrades in response to trypsin.

17. A method of fabricating a biodegradable sensor, comprising:
providing a substrate;
depositing an electrode layer on the substrate, wherein the electrode layer comprises a split-ring resonator and at least one interdigitated capacitor connected in series with the split-ring resonator;
depositing a passivation layer over the electrode layer; and
depositing a sensing film over the passivation layer, wherein the sensing film is configured to change in thickness in response to an analyte of interest.

18. The method of claim 17, wherein providing the substrate comprises spin coating a mechanically stable biodegradable polymer onto a support layer.

19. The method of claim 18, wherein depositing the electrode layer comprises depositing a biodegradable metal and patterning the biodegradable metal to form the split-ring resonator and the at least one interdigitated capacitor.

20. The method of claim 17, wherein depositing the sensing film comprises depositing a pH-sensitive polymer comprising a poly(meth)acrylate copolymer comprising units of methacrylic acid and methyl methacrylate.
